(19) United States
(12) Patent Application Publication
(10) Pub. No.: US 2005/0004199 A1
(43) Pub. Date: Jan. 6, 2005

(54) Treatment of inflammation with a combination of a cyclooxygenase-2 inhibitor and an integrin alpha-v antagonist

(76) Inventors: George Hartman, Lansdale, PA (US); Mark Duggan, Schwenksville, PA (US); Gideon Rodan, Bryn Mawr, PA (US); Sevgi B. Rodan, Bryn Mawr, PA (US); Le T. Duong, Lansdale, PA (US); Donald B. Kimmel, Doylestown, PA (US)

Correspondence Address: MERCK AND CO INC, P O BOX 2000, RAHWAY, NJ, 070650907, US

(21) Appl. No.: 10/380,785

(22) PCT Filed: Sep. 14, 2001
(86) PCT No.: PCT/US01/42146

Related U.S. Application Data
(60) Provisional application No. 60/233,609, filed on Sep. 18, 2000.

(51) Int. Cl. A61K 31/415; A61K 31/365

(52) U.S. Cl. 514/406; 514/473

(57) ABSTRACT

The present invention provides for methods for treating or preventing an inflammatory disease or condition in a mammalian patient in need of such treatment comprising administering to said patient a cyclooxygenase-2 specific inhibitor in combination with an α_{V}β₃, ^{α}V^{β}5? and/or α_{V}β₆ integrin receptor antagonist in an amount effective to treat or prevent the inflammatory disease or condition. The present invention also provides for pharmaceutical compositions for the treatment or prevention of an inflammatory disease or condition. Further, the invention provides for the manufacture of a medicament useful in the treatment or prevention of an inflammatory disease or condition.

### BACKGROUND OF THE INVENTION

Non-steroidal, antiinflammatory drugs exert most of their antiinflammatory, analgesic and antipyretic activity through inhibition of prostaglandin G/H synthase, also known as cyclooxygenase. Initially, only one form of cyclooxygenase was known, this corresponding to cyclooxygenase-1 (COX-1) or the constitutive enzyme, as originally identified in bovine seminal vesicles. More recently the gene for a second inducible form of cyclooxygenase, cyclooxygenase-2 (COX-2) has been cloned, sequenced and characterized initially from chicken, murine and human sources. This enzyme is distinct from the COX-1 which has been cloned, sequenced and characterized from various sources including the sheep, the mouse and man. The second form of cyclooxygenase, COX-2, is rapidly and readily inducible by a number of agents including mitogens, endotoxin, hormones, cytokines and growth factors. As prostaglandins have both physiological and pathological roles, we have concluded that the constitutive enzyme, COX-1, is responsible, in large part, for endogenous basal release of prostaglandins and hence is important in their physiological functions such as the maintenance of gastrointestinal integrity and renal blood flow. In contrast, the inducible form, COX-2, is mainly responsible for the pathological effects of prostaglandins where rapid induction of the enzyme would occur in response to such agents as inflammatory agents, hormones, growth factors, and cytokines. Thus, a selective inhibitor of COX-2 has similar antiinflammatory, antipyretic and analgesic properties to a conventional non-steroidal antiinflammatory drug, but has diminished ability to induce some of the mechanism-based side effects. In particular, such a compound has a reduced potential for gastrointestinal toxicity, a reduced potential for renal side effects, a reduced effect on bleeding times and possibly a lessened ability to induce asthma attacks in aspirin-sensitive asthmatic subjects.

Integrin receptors are heterodimeric transmembrane receptors through which cells attach and communicate with extracellular matrices and other cells (See S. B. Rodan and G. A. Rodan, “Integrin Function In Osteoclasts”, Journal of Endocrinology, Vol. 154, S47-S56 (1997)). α_{V}β₃ integrin receptor antagonists are useful for inhibiting bone resorption, which is mediated by the action of cells known as osteoclasts. Osteoclasts are large multinucleated cells of up to about 100 μm in diameter that resorb mineralized tissue, containing chiefly calcium phosphate in vertebrates. Osteoclasts are actively motile cells that migrate along the surface of bone, and can adhere to bone, secrete necessary acids and proteases, thereby causing the actual resorption of mineralized tissue. More specifically, osteoclasts are believed to exist in at least two physiological states, namely, the secretory state and the migratory or motile state. In the secretory state, osteoclasts are flat, attach to the bone matrix via a tight attachment zone (sealing zone), become highly polarized, form a ruffled border, and secrete lysosomal enzymes and protons to resorb bone. The adhesion of osteoclasts to bone surfaces is an important initial step in bone resorption. In the migratory or motile state, the osteoclasts migrate across bone matrix and do not take part in resorption until they again attach to bone.

Integrin-mediated adhesion is important for osteoclast attachment, activation and migration. The most abundant integrin in osteoclasts, e.g., in rat, chicken, mouse and human osteoclasts, is the α_{V}β₃ integrin receptor, which is thought to interact in bone with matrix proteins that contain the RGD sequence. Antibodies to α_{V}β₃ block bone resorption in vitro indicating that this integrin plays a key role in the resorptive process. There is increasing evidence to suggest that α_{V}β₃ ligands can be used effectively to inhibit osteoclast mediated bone resorption in vivo in mammals.

The current major bone diseases of public concern are osteoporosis, hypercalcemia of malignancy, osteopenia due to bone metastases, periodontal disease, hyperparathyroidism, periarticular erosions in rheumatoid arthritis, Paget's disease, immobilization-induced osteopenia, and glucocorticoid-induced osteoporosis. All of these conditions are characterized by bone loss, resulting from an imbalance between bone resorption, i.e. breakdown, and bone formation, which continues throughout life at the rate of about 14% per year on the average. However, the rate of bone turnover differs from site to site; for example, it is higher in the trabecular bone of the vertebrae and the alveolar bone in the jaws than in the cortices of the long bones. The potential for bone loss is directly related to turnover and can amount to over 5% per year in vertebrae immediately following menopause, a condition which leads to increased fracture risk.

In a recent study, α_{V}β₃ integrin receptor antagonists have been shown to be effective against arthritic disease induced in rabbits (See Storgard et al., “Decreased Angiogenesis and Arthritic Disease in Rabbits Treated with an α_{V}β₃ Antagonist,” J. Clin. Invest. 103, 47-54 (1999)).

Another class of integrin receptor antagonists inhibit neovascularization by acting as antagonists of the integrin receptor, α_{V}β₅. A monoclonal antibody for α_{V}β₅ has been shown to inhibit VEGF-induced angiogenesis in rabbit cornea and the chick chorioallantoic membrane model (See M. C. Friedlander, et al., Science 270, 1500-1502, (1995)). Thus, compounds that antagonize α_{V}β₅ could be useful for treating and preventing macular degeneration, diabetic retinopathy, tumor growth, and metastasis.

Additionally, a class of integrin receptor antagonists can inhibit inflammation by acting as antagonists of the integrin receptor, α_{V}β₆, which is expressed in migrating keratinocytes during the later stages of wound healing and remains expressed until the wound is closed (See Christofidou-Solomidou, et al., “Expression and Function of Endothelial Cell α_{V} Integrin Receptors in Wound-Induced Human Angiogenesis in Human Skin/SCID Mice Chimeras,” American Journal of Pathology, Vol. 151, No. 4, pp. 975-983 (October 1997)). α_{V}β₆ participates in the modulation of epithelial inflammation and is induced in response to local injury or inflammation (See Xiao-Zhu Huang, et al., “Inactivation of the Integrin β₆ Subunit Gene Reveals a Role of Epithelial Integrins in Regulating Inflammation in the Lungs and Skin,” Journal of Cell Biology, Vol. 133, No. 4, pp. 921-928 May 1996)).

In addition, certain integrin receptor antagonist compounds antagonize both the α_{V}β₃ and α_{V}β₅ receptors. These compounds, referred to as “dual α_{V}β₃/α_{V}β₅ antagonists,” are useful for inhibiting bone resorption, treating and preventing osteoporosis, and inhibiting vascular restenosis, diabetic retinopathy, macular degeneration, angiogenesis, atherosclerosis, inflammation (rheumatoid arthritis), tumor growth, and metastasis.

In addition, certain integrin receptor antagonist compounds are useful as mixed α_{V}β₃, α_{V}β₅, and α_{V}β₆ receptor antagonists.

The instant invention provides for a combination therapy comprised of an α_{V}β₃, α_{V}β₅ and/or α_{V}β₆ receptor antagonist and a cyclooxygenase-2 specific inhibitor for the prevention and treatment of an inflammatory disease or condition. When administered as part of a combination therapy, the cyclooxygenase-2 specific inhibitor together with the integrin α_{V} antagonist provides enhanced therapy treatment options as compared to the administration of either the cyclooxygenase-2 inhibitor or the integrin α_{V} antagonist alone.

### SUMMARY OF THE INVENTION

The present invention provides for methods for treating or preventing an inflammatory disease or condition in a mammalian patient in need of such treatment comprising administering to said patient a cycloxoygenase-2 specific inhibitor in combination with an α_{V}β₃, α_{V}β₅ and/or α_{V}β₆ integrin receptor antagonist in an amount effective to treat or prevent the inflammatory disease or condition. The present invention also provides for pharmaceutical compositions for the treatment or prevention of an inflammatory disease or condition. Further, the invention provides for the manufacture of a medicament useful in the treatment or prevention of an inflammatory disease or condition.

### DETAILED DESCRIPTION OF THE INVENTION

The present invention is concerned with the combination of a cyclooxygenase-2 specific inhibitor with an α_{V}β₃, α_{V}β₅ and/or α_{V}β₆ integrin receptor antagonist for the treatment or prevention of an inflammatory disease or condition. This particular combination produces superior results in the treatment of an inflammatory disease or condition compared to the results from administering a cyclooxygenase-2 inhibitor or an α_{V}β₃, α_{V}β₅ and/or α_{V}β₆ integrin receptor antagonist alone. It is an object of the invention to describe the combination of the two drugs in the treatment of an inflammatory disease or condition. In addition, it is an object of the instant invention to describe preferred embodiments within each category of compounds that are used as elements in the instant combination. It is a further object of this invention to describe pharmaceutical compositions for the treatment or prevention of an inflammatory disease or condition. It is a still further object of this invention to provide a method of manufacture of a medicament containing the present drug combination that is useful for the treatment of an inflammatory disease or condition. Further objects will become apparent from a reading of the following description.

The invention encompasses a method for treating or preventing an inflammatory disease or condition in a mammalian patient in need of such treatment comprising administering to said patient an integrin α_{V} antagonist in combination with a cyclooxygenase-2 specific inhibitor in an amount that is effective to treat or prevent the inflammatory disease or condition.

In an embodiment of the invention, the cyclooxygenase-2 specific inhibitor is selected from the group consisting of:
(1) 3-phenyl-4-(4-(methylsulfonyl)phenyl)-2-(5H)-furanone;
(2) 3-(3-fluorophenyl)-4-(4-methylsulfonyl)phenyl)-2-(5H)-furanone;
(3) 3-(3,4-difluorophenyl)-4-(4-(methylsulfonyl)phenyl)-2-(5H)-furanone;
(4) 3-(3,4-trichlorophenyl)-4-(4-(methylsulfonyl)phenyl)-2-(5H)-furanone;
(5) 3-(3,4-dichlorophenyl)-4-(4-(aminosulfonyl)phenyl)-2-(5H)-furanone;
(6) 3-(3-chloro-4-methoxyphenyl)-4-(4-aminosulfonyl)phenyl)-2-(5H)-furanone;
(7) 4-[5-(4-methylphenyl)-3-(trifluoromethyl)-1H-pyrazol-1-yl]benzenesulfonamide;
(8) (5S)-ethyl-5-methyl-4-(4-(methanesulfonyl)phenyl)-3-(2-propoxy)-(5H)-furan-2-one;
(9) 5,5-dimethyl-4-(4-(methylsulfonyl)phenyl)-3-(2-propoxy)-5H-furan-2-one;
(10) 5,5-dimethyl-4-(4-(methylsulfonyl)phenyl)-3-(5-bromopyridin-2-yloxy)-5H-furan-2-one;
(11) 5-methyl-4-(4-methylsulfonyl)phenyl)-3-(2-(propoxy)-5-(2-trifluoroethyl)-5H-furan-2-one;
(12) 3-(3-trifluoromethyl)phenoxy-4-(4-methylsulfonyl)phenyl)-5,5-dimethyl-5H-furan-2-one;
(13) (5R)-3-(3-chloro-4-methoxyphenoxy)-5-ethyl-5-methyl-4-(4-methylsulfonyl)phenyl-5H-furan-2-one;
(14) 5-chloro-3-(4-methylsulfonyl)phenyl-2-(2-methyl-5-pyridinyl)pyridine;
(15) 5-chloro-3-(4-methylsulfonyl)phenyl-2-(2-ethyl-5-pyridinyl)pyridine;
(16) 5-chloro-3-(4-(methylsulfonyl)phenyl)-2-(3-pyridinyl)pyridine;
(17) 4-(5-methyl-3-phenyl-4-isoxazolyl)-benzenesulfonamide; and
(18) N-[[4-(5-methyl-3-phenyl-4-isoxazolyl)phenyl]sulfonyl]propanamide;
   or a pharmaceutically acceptable salt thereof.

In another embodiment of the invention, the cyclooxygenase-2 specific inhibitor is selected from the group consisting of:
(1) 3-phenyl-4-(4-(methylsulfonyl)phenyl)-2-(5H)-furanone;
(2) 4-[5-(4-methylphenyl)-3-(trifluoromethyl)-1H-pyrazol-1-yl]benzenesulfonamide;
(3) 4-(5-methyl-3-phenyl-4-isoxazolyl)-benzenesulfonamide;
(4) N-[[4-(5-methyl-3-phenyl-4-isoxazolyl)phenyl]sulfonyl]propanamide;
(5) 5-chloro-3-(4-methylsulfonyl)phenyl-2-(2-methyl-5-pyridinyl)pyridine; and
(6) (5S)-ethyl-5-methyl-4-(4-(methanesulfonyl)phenyl)-3-(2-propoxy)-(5H)-furan-2-one;
   or a pharmaceutically acceptable salt thereof.

In another embodiment of the invention, the integrin α_{V} antagonist is a compound of formula I 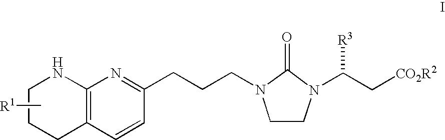
or a pharmaceutically acceptable salt thereof, wherein:
R¹ is hydrogen or C₁₋₄ alkyl;
R² is hydrogen or C₁₋₄ alkyl; and
R³ is selected from the group consisting of: dihydrobenzofuranyl, phenyl, quinolinyl and pyridinyl, each optionally substituted with 1-2 substituents independently selected from the group consisting of: halo, hydroxy, cyano, C₁₋₆alkyl, C₁₋₃alkoxy, amino, C₁₋₃ alkylamino and di(C₁₋₃) alkylamino, said C₁₋₆alkyl and C₁₋₃alkoxy each optionally substituted with 1-3 halo groups.

In another embodiment of the invention, the integrin α_{V} antagonist is selected from the group consisting of:
(1) 3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;
(2) 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid;
(3) 3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid;
(4) 3(S)-(quinolin-3-yl)-3-{2-oxo-3-[3-(5,6,7,8-tetrahydro-[1,8]naphthyridin-2-yl)-propyl]-imidazolidin-1-yl}-propionic acid; and
(5) 3(S)-(4-ethoxy-3-fluorophenyl)-3-{2-oxo-3-[3-(5,6,7,8-tetrahydro-[1,8]-naphthyridin-2-yl)-propyl]-imidazolidin-1-yl}-propionic acid;
   or a pharmaceutically acceptable salt thereof.

In a subclass of this embodiment, the cyclooxygenase-2 specific inhibitor is selected from the group consisting of:
(1) 3-phenyl-4-(4-(methylsulfonyl)phenyl)-2-(5H)-furanone;
(2) 4-[5-(4-methylphenyl)-3-(trifluoromethyl)-1H-pyrazol-1-yl]benzenesulfonamide;
(3) 4-(5-methyl-3-phenyl-4-isoxazolyl)-benzenesulfonamide;
(4) N-[[4-(5-methyl-3-phenyl-4-isoxazolyl)phenyl]sulfonyl]propanamide;
(5) 5-chloro-3-(4-methylsulfonyl)phenyl-2-(2-methyl-5-pyridinyl)pyridine; and
(6) (5S)-ethyl-5-methyl-4-(4-(methanesulfonyl)phenyl)-3-(2-propoxy)-(5H)-furan-2-one;
   or a pharmaceutically acceptable salt thereof.

In another subclass of this embodiment, the integrin α_{V} antagonist is 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid or a pharmaceutically acceptable salt thereof. Within this subclass is the method wherein the cyclooxygenase-2 specific inhibitor is selected from the group consisting of: 3-phenyl-4-(4-(methylsulfonyl)phenyl)-2-(5H)-furanone, 5-chloro-3-(4-methylsulfonyl)phenyl-2-(2-methyl-5-pyridinyl)pyridine, and (5S)-ethyl-5-methyl-4-(4-(methanesulfonyl)phenyl)-3-(2-propoxy)-(5H)-furan-2-one, or a pharmaceutically acceptable salt thereof.

In another embodiment of the invention, the integrin α_{V} antagonist is a compound of formula II 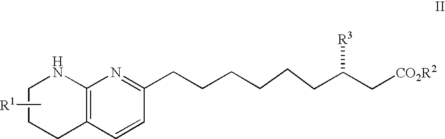
or a pharmaceutically acceptable salt thereof, wherein:
R¹ is hydrogen or C₁₋₄ alkyl;
R² is hydrogen or C₁₋₄ alkyl; and
R³ is selected from the group consisting of: quinolinyl, pyridinyl and pyrimidinyl, each optionally substituted with 1-2 substituents independently selected from the group consisting of: halo, hydroxy, cyano, C₁₋₆alkyl, C₁₋₃alkoxy, amino, C₁₋₃alkylamino and di(C₁₋₃)alkylamino, said C₁₋₆alkyl and C₁₋₃alkoxy each optionally substituted with 1-3 halo groups.

In another embodiment of the invention, the integrin α_{V} antagonist is selected from the group consisting of:
(1) 3-(pyridin-3-yl)-9-(5,6,7,8-tetrahydro-[1,8]-naphthyridin-2-yl)-nonanoic acid;
(2) 3(S)-(6-methoxy-pyridin-3-yl)-9-(5,6,7,8-tetrahydro-[1,8]-naphthyridin-2-yl)-nonanoic acid;
(3) 3(S)-(pyrimidin-5-yl)-9-(5,6,7,8-tetrahydro-[1,81-naphthyridin-2-yl)-nonanoic acid;
(4) 3(S)-(6-amino-pyridin-3-yl)-9-(5,6,7,8-tetrahydro-[1,8]-naphthyridin-2-yl)-nonanoic acid;
(5) 3(S)-(2-methyl-pyrimidin-5-yl)-9-(5,6,7,8-tetrahydro-[1,8]-naphthyridin-2-yl)-nonanoic acid; and
(6) 3(S)-(quinolin-3-yl)-9-(5,6,7,8-tetrahydro-[1,8]-naphthyridin-2-yl)-nonanoic acid;
   or a pharmaceutically acceptable salt thereof.

In a subclass of this embodiment, the cyclooxygenase-2 specific inhibitor is selected from the group consisting of:
(1) 3-phenyl-4-(4-(methylsulfonyl)phenyl)-2-(5H)-furanone;
(2) 4-[5-(4-methylphenyl)-3-(trifluoromethyl)-1H-pyrazol-1-yl]benzenesulfonamide;
(3) 4-(5-methyl-3-phenyl-4-isoxazolyl)-benzenesulfonamide;
(4) N-[[4-(5-methyl-3-phenyl-4-isoxazolyl)phenyl]sulfonyl]propanamide;
(5) 5-chloro-3-(4-methylsulfonyl)phenyl-2-(2-methyl-5-pyridinyl)pyridine; and
(6) (5S)-ethyl-5-methyl-4-(4-(methanesulfonyl)phenyl)-3-(2-propoxy)-(5H)-furan-2-one;
   or a pharmaceutically acceptable salt thereof.

In another subclass of this embodiment, the integrin α_{V} antagonist is selected from the group consisting of: 3(S)-(pyrimidin-5-yl)-9-(5,6,7,8-tetrahydro-[1,8]-naphthyridin-2-yl)-nonanoic acid and 3(S)-(2-methyl-pyrimidin-5-yl)-9-(5,6,7,8-tetrahydro-[1,8]-naphthyridin-2-yl)-nonanoic acid, or a pharmaceutically acceptable salt thereof. Within this subclass is the method wherein the cyclooxygenase-2 specific inhibitor is selected from the group consisting of: 3-phenyl-4-(4-(methylsulfonyl)phenyl)-2-(5H)-furanone, 5-chloro-3-(4-methylsulfonyl)phenyl-2-(2-methyl-5-pyridinyl)pyridine, and (5S)-ethyl-5-methyl-4-(4-(methanesulfonyl)phenyl)-3-(2-propoxy)-(5H)-furan-2-one, or a pharmaceutically acceptable salt thereof.

In an embodiment of the invention that is of interest, the integrin α_{V} antagonist is an α_{V}β₃ integrin receptor antagonist. In another embodiment, the integrin α_{V} antagonist is an α_{V}β₅ integrin receptor antagonist. In another embodiment, the integrin α_{V} antagonist is an α_{V}β₆ integrin receptor antagonist. In another embodiment, the integrin α_{V} antagonist is a dual α_{V}β₃/α_{V}β₅ integrin receptor antagonist. In another embodiment, the integrin α_{V} antagonist is a mixed α_{V}β₃, α_{V}β₅ and α_{V}β₆ integrin receptor antagonist.

An embodiment of the invention encompasses the use of an integrin α_{V} antagonist in combination with a cyclooxygenase-2 specific inhibitor for the preparation of a medicament useful for the treatment or prevention of an inflammatory disease or condition.

Another embodiment encompasses a method for treating or preventing rheumatoid arthritis in a mammalian patient in need of such treatment comprising administering to said patient an integrin α_{V} antagonist in combination with a cyclooxygenase-2 specific inhibitor in an amount that is effective to treat or prevent rheumatoid arthritis.

In a subclass of this embodiment, the cyclooxygenase-2 specific inhibitor is selected from the group consisting of:
(1) 3-phenyl-4-(4-(methylsulfonyl)phenyl)-2-(5H)-furanone;
(2) 4-[5-(4-methylphenyl)-3-(trifluoromethyl)-1H-pyrazol-1-yl]benzenesulfonamide;
(3) 4-(5-methyl-3-phenyl-4-isoxazolyl)-benzenesulfonamide;
(4) N-[[4-(5-methyl-3-phenyl-4-isoxazolyl)phenyl]sulfonyl]propanamide;
(5) 5-chloro-3-(4-methylsulfonyl)phenyl-2-(2-methyl-5-pyridinyl)pyridine; and
(6) (5S)-ethyl-5-methyl-4-(4-(methanesulfonyl)phenyl)-3-(2-propoxy)-(5H)-furan-2-one;
   or a pharmaceutically acceptable salt thereof.

In another subclass of this embodiment, the integrin α_{V} antagonist is selected from the group consisting of:
(1) 3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;
(2) 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid;
(3) 3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid;
(4) 3(S)-(quinolin-3-yl)-3-{2-oxo-3-[3-(5,6,7,8-tetrahydro-[1,8]naphthyridin-2-yl)-propyl]-imidazolidin-1-yl}-propionic acid;
(5) 3(S)-(4-ethoxy-3-fluorophenyl)-3-{2-oxo-3-[3-(5,6,7,8-tetrahydro-[1,8]-naphthyridin-2-yl)-propyl]-imidazolidin-1-yl}-propionic acid;
(6) 3-(pyridin-3-yl)-9-(5,6,7,8-tetrahydro-[1,8]-naphthyridin-2-yl)-nonanoic acid;
(7) 3(S)-(6-methoxy-pyridin-3-yl)-9-(5,6,7,8-tetrahydro-[1,8]-naphthyridin-2-yl)-nonanoic acid;
(8) 3(S)-(pyrimidin-5-yl)-9-(5,6,7,8-tetrahydro-[1,8]-naphthyridin-2-yl)-nonanoic acid;
(9) 3(S)-(6-amino-pyridin-3-yl)-9-(5,6,7,8-tetrahydro-[1,8]-naphthyridin-2-yl)-nonanoic acid;
(10) 3(S)-(2-methyl-pyrimidin-5-yl)-9-(5,6,7,8-tetrahydro-[1,8]-naphthyridin-2-yl)-nonanoic acid; and
(11) 3(S)-(quinolin-3-yl)-9-(5,6,7,8-tetrahydro-[1,8]-naphthyridin-2-yl)-nonanoic acid;
   or a pharmaceutically acceptable salt thereof.

Another embodiment encompasses a method for treating or preventing osteoarthritis in a mammalian patient in need of such treatment comprising administering to said patient an integrin α_{V} antagonist in combination with a cyclooxygenase-2 specific inhibitor in an amount that is effective to treat or prevent osteoarthritis.

In a subclass of this embodiment, the cyclooxygenase-2 specific inhibitor is selected from the group consisting of:
(1) 3-phenyl-4-(4-(methylsulfonyl)phenyl)-2-(5H)-furanone;
(2) 4-[5-(4-methylphenyl)-3-(trifluoromethyl)-1H-pyrazol-1-yl]benzenesulfonamide;
(3) 4-(5-methyl-3-phenyl-4-isoxazolyl)-benzenesulfonamide;
(4) N-[[4-(5-methyl-3-phenyl-4-isoxazolyl)phenyl]sulfonyl]propanamide;
(5) 5-chloro-3-(4-methylsulfonyl)phenyl-2-(2-methyl-5-pyridinyl)pyridine; and
(6) (5S)-ethyl-5-methyl-4-(4-(methanesulfonyl)phenyl)-3-(2-propoxy)-(5H)-furan-2-one;
   or a pharmaceutically acceptable salt thereof.

In another subclass of this embodiment, the integrin α_{V} antagonist is selected from the group consisting of:
(1) 3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;
(2) 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid;
(3) 3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid;
(4) 3(S)-(quinolin-3-yl)-3-{2-oxo-3-[3-(5,6,7,8-tetrahydro-[1,8]naphthyridin-2-yl)-propyl]-imidazolidin-1-yl}-propionic acid;
(5) 3(S)-(4-ethoxy-3-fluorophenyl)-3-{2-oxo-3-[3-(5,6,7,8-tetrahydro-[1,8]-naphthyridin-2-yl)-propyl]-imidazolidin-1-yl}-propionic acid;
(6) 3-(pyridin-3-yl)-9-(5,6,7,8-tetrahydro-[1,8]-naphthyridin-2-yl)-nonanoic acid;
(7) 3(S)-(6-methoxy-pyridin-3-yl)-9-(5,6,7,8-tetrahydro-[1,8]-naphthyridin-2-yl)-nonanoic acid;
(8) 3(S)-(pyrimidin-5-yl)-9-(5,6,7,8-tetrahydro-[1,8]-naphthyridin-2-yl)-nonanoic acid;
(9) 3(S)-(6-amino-pyridin-3-yl)-9-(5,6,7,8-tetrahydro-[1,8]-naphthyridin-2-yl)-nonanoic acid;
(10) 3(S)-(2-methyl-pyrimidin-5-yl)-9-(5,6,7,8-tetrahydro-[1,8]-naphthyridin-2-yl)-nonanoic acid; and
(11) 3(S)-(quinolin-3-yl)-9-(5,6,7,8-tetrahydro-[1,8]-naphthyridin-2-yl)-nonanoic acid;
   or a pharmaceutically acceptable salt thereof.

The invention also encompasses a pharmaceutical composition comprising a pharmaceutically acceptable carrier, a cyclooxygenase-2 specific inhibitor and an integrin α_{V} antagonist of formula I 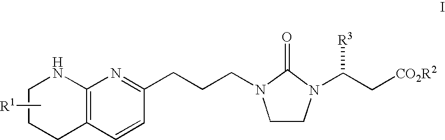
or a pharmaceutically acceptable salt thereof, wherein:
R¹ is hydrogen or C₁₋₄ alkyl;
R² is hydrogen or C₁₋₄ alkyl; and
R³ is selected from the group consisting of: dihydrobenzofuranyl, phenyl, quinolinyl and pyridinyl, each optionally substituted with 1-2 substituents independently selected from the group consisting of: halo, hydroxy, cyano, C₁₋₆alkyl, C₁₋₃alkoxy, amino, C₁₋₃ alkylamino and di(C₁₋₃)alkylamino, said C₁₋₆alkyl and C₁₋₃alkoxy each optionally substituted with 1-3 halo groups.

An embodiment of the invention encompasses the pharmaceutical composition of formula I wherein the cyclooxygenase-2 specific inhibitor is selected from the group consisting of:
(1) 3-phenyl-4-(4-(methylsulfonyl)phenyl)-2-(5H)-furanone;
(2) 3-(3-fluorophenyl)-4-(4-methylsulfonyl)phenyl)-2-(5H)-furanone;
(3) 3-(3,4-difluorophenyl)-4-(4-(methylsulfonyl)phenyl)-2-(5H)-furanone;
(4) 3-(3,4-trichlorophenyl)-4-(4-(methylsulfonyl)phenyl)-2-(5H)-furanone;
(5) 3-(3,4-dichlorophenyl)-4-(4-(aminosulfonyl)phenyl)-2-(5H)-furanone;
(6) 3-(3-chloro-4-methoxyphenyl)-4-(4-aminosulfonyl)phenyl)-2-(5H)-furanone;
(7) 4-[5-(4-methylphenyl)-3-(trifluoromethyl)-1H-pyrazol-1-yl]benzenesulfonamide;
(8) (5S)-ethyl-5-methyl-4-(4-(methanesulfonyl)phenyl)-3-(2-propoxy)-(5H)-furan-2-one;
(9) 5,5-dimethyl-4-(4-(methylsulfonyl)phenyl)-3-(2-propoxy)-5H-furan-2-one;
(10) 5,5-dimethyl-4-(4-(methylsulfonyl)phenyl)-3-(5-bromopyridin-2-yloxy)-5H-furan-2-one;
(11) 5-methyl-4-(4-methylsulfonyl)phenyl)-3-(2-(propoxy)-5-(2-trifluoroethyl)-5H-furan-2-one;
(12) 3-(3-trifluoromethyl)phenoxy-4-(4-methylsulfonyl)phenyl)-5,5-dimethyl-5H-furan-2-one;
(13) (5R)-3-(3-chloro-4-methoxyphenoxy)-5-ethyl-5-methyl-4-(4-methylsulfonyl)phenyl-5H-furan-2-one;
(14) 5-chloro-3-(4-methylsulfonyl)phenyl-2-(2-methyl-5-pyridinyl)pyridine;
(15) 5-chloro-3-(4-methylsulfonyl)phenyl-2-(2-ethyl-5-pyridinyl)pyridine;
(16) 5-chloro-3-(4-(methylsulfonyl)phenyl)-2-(3-pyridinyl)pyridine;
(17) 4-(5-methyl-3-phenyl-4-isoxazolyl)-benzenesulfonamide; and
(18) N-[[4-(5-methyl-3-phenyl-4-isoxazolyl)phenyl]sulfonyl]propanamide;
   or a pharmaceutically acceptable salt thereof.

Another embodiment of the invention encompasses the pharmaceutical composition of formula I wherein the integrin α_{V} antagonist is selected from the group consisting of:
(1) 3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;
(2) 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid;
(3) 3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid;
(4) 3(S)-(quinolin-3-yl)-3-{2-oxo-3-[3-(5,6,7,8-tetrahydro-[1,8]naphthyridin-2-yl)-propyl]-imidazolidin-1-yl}-propionic acid; and
(5) 3(S)-(4-ethoxy-3-fluorophenyl)-3-{2-oxo-3-[3-(5,6,7,8-tetrahydro-[1,8]-naphthyridin-2-yl)-propyl]-imidazolidin-1-yl}-propionic acid;
   or a pharmaceutically acceptable salt thereof.

A subclass of this embodiment encompasses the pharmaceutical composition wherein the integrin α_{V} antagonist is 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid or a pharmaceutically acceptable salt thereof. Within this subclass is the pharmaceutical composition wherein the cyclooxygenase-2 specific inhibitor is selected from the group consisting of: 3-phenyl-4-(4-(methylsulfonyl)phenyl)-2-(5H)-furanone, 5-chloro-3-(4-methylsulfonyl)phenyl-2-(2-methyl-5-pyridinyl)pyridine, and (5S)-ethyl-5-methyl-4-(4-(methanesulfonyl)phenyl)-3-(2-propoxy)-(5H)-furan-2-one, or a pharmaceutically acceptable salt thereof.

In another embodiment, the invention encompasses a pharmaceutical composition comprising a pharmaceutically acceptable carrier, a cyclooxygenase-2 specific inhibitor and an integrin α_{V} antagonist of formula II 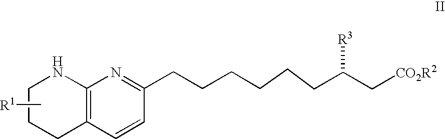
or a pharmaceutically acceptable salt thereof, wherein:
R¹ is hydrogen or C₁₋₄ alkyl;
R² is hydrogen or C₁₋₄ alkyl; and
R³ is selected from the group consisting of: quinolinyl, pyridinyl and pyrimidinyl, each optionally substituted with 1-2 substituents independently selected from the group consisting of: halo, hydroxy, cyano, C₁₋₆alkyl, C₁₋₃alkoxy, amino, C₁₋₃ alkylamino and di(C₁₋₃) alkylamino, said C₁₋₆alkyl and C₁₋₃alkoxy each optionally substituted with 1-3 halo groups.

Another embodiment of the invention encompasses the pharmaceutical composition of formula II wherein the cyclooxygenase-2 specific inhibitor is selected from the group consisting of:
(1) 3-phenyl-4-(4-(methylsulfonyl)phenyl)-2-(5H)-furanone;
(2) 3-(3-fluorophenyl)-4-(4-methylsulfonyl)phenyl)-2-(5H)-furanone;
(3) 3-(3,4-difluorophenyl)-4-(4-(methylsulfonyl)phenyl)-2-(5H)-furanone;
(4) 3-(3,4-trichlorophenyl)-4-(4-(methylsulfonyl)phenyl)-2-(5H)-furanone;
(5) 3-(3,4-dichlorophenyl)-4-(4-(aminosulfonyl)phenyl)-2-(5H)-furanone;
(6) 3-(3-chloro-4-methoxyphenyl)-4-(4-aminosulfonyl)phenyl)-2-(5H)-furanone;
(7) 4-[5-(4-methylphenyl)-3-(trifluoromethyl)-1H-pyrazol-1-yl]benzenesulfonamide;
(8) (5S)-ethyl-5-methyl-4-(4-(methanesulfonyl)phenyl)-3-(2-propoxy)-(5H)-furan-2-one;
(9) 5,5-dimethyl-4-(4-(methylsulfonyl)phenyl)-3-(2-propoxy)-5H-furan-2-one;
(10) 5,5-dimethyl-4-(4-(methylsulfonyl)phenyl)-3-(5-bromopyridin-2-yloxy)-5H-furan-2-one;
(11) 5-methyl-4-(4-methylsulfonyl)phenyl)-3-(2-(propoxy)-5-(2-trifluoroethyl)-5H-furan-2-one;
(12) 3-(3-trifluoromethyl)phenoxy-4-(4-methylsulfonyl)phenyl)-5,5-dimethyl-5H-furan-2-one;
(13) (5R)-3-(3-chloro-4-methoxyphenoxy)-5-ethyl-5-methyl-4-(4-methylsulfonyl)phenyl-5H-furan-2-one;
(14) 5-chloro-3-(4-methylsulfonyl)phenyl-2-(2-methyl-5-pyridinyl)pyridine;
(15) 5-chloro-3-(4-methylsulfonyl)phenyl-2-(2-ethyl-5-pyridinyl)pyridine;
(16) 5-chloro-3-(4-(methylsulfonyl)phenyl)-2-(3-pyridinyl)pyridine;
(17) 4-(5-methyl-3-phenyl-4-isoxazolyl)-benzenesulfonamide; and
(18) N-[[4-(5-methyl-3-phenyl-4-isoxazolyl)phenyl]sulfonyl]propanamide;
   or a pharmaceutically acceptable salt thereof.

Another embodiment of the invention encompasses the pharmaceutical composition of formula II wherein the integrin α_{V} antagonist is selected from the group consisting of:
(1) 3-(pyridin-3-yl)-9-(5,6,7,8-tetrahydro-[1,8]-naphthyridin-2-yl)-nonanoic acid;
(2) 3(S)-(6-methoxy-pyridin-3-yl)-9-(5,6,7,8-tetrahydro-[1,8]-naphthyridin-2-yl)-nonanoic acid;
(3) 3(S)-(pyrimidin-5-yl)-9-(5,6,7,8-tetrahydro-[1,8]-naphthyridin-2-yl)-nonanoic acid;
(4) 3(S)-(6-amino-pyridin-3-yl)-9-(5,6,7,8-tetrahydro-[1,8]-naphthyridin-2-yl)-nonanoic acid;
(5) 3(S)-(2-methyl-pyrimidin-5-yl)-9-(5,6,7,8-tetrahydro-[1,8]-naphthyridin-2-yl)-nonanoic acid; and
(6) 3(S)-(quinolin-3-yl)-9-(5,6,7,8-tetrahydro-[1,8]-naphthyridin-2-yl)-nonanoic acid;
   or a pharmaceutically acceptable salt thereof.

A subclass of this embodiment encompasses the pharmaceutical composition wherein the integrin α_{V} antagonist is selected from the group consisting of: 3(S)-(pyrimidin-5-yl)-9-(5,6,7,8-tetrahydro-[1,8]-naphthyridin-2-yl)-nonanoic acid and 3(S)-(2-methyl-pyrimidin-5-yl)-9-(5,6,7,8-tetrahydro-[1,8]-naphthyridin-2-yl)-nonanoic acid, or a pharmaceutically acceptable salt thereof. Within this subclass is the pharmaceutical composition wherein the cyclooxygenase-2 specific inhibitor is selected from the group consisting of: 3-phenyl-4-(4-(methylsulfonyl)phenyl)-2-(5H)-furanone, 5-chloro-3-(4-methylsulfonyl)phenyl-2-(2-methyl-5-pyridinyl)pyridine, and (5S)-ethyl-5-methyl-4-(4-(methanesulfonyl)phenyl)-3-(2-propoxy)-(5H)-furan-2-one, or a pharmaceutically acceptable salt thereof.

The present invention also encompasses a method for treating or preventing an inflammatory disease or condition in a mammalian patient in need of such treatment comprising administering to said patient an integrin α_{V} antagonist selected from the group consisting of:
(1) 3-(pyridin-3-yl)-9-(5,6,7,8-tetrahydro-[1,8]-naphthyridin-2-yl)-nonanoic acid,
(2) 3(S)-(6-methoxy-pyridin-3-yl)-9-(5,6,7,8-tetrahydro-[1,8]-naphthyridin-2-yl)-nonanoic acid,
(3) 3(S)-(pyrimidin-5-yl)-9-(5,6,7,8-tetrahydro-[1,8]-naphthyridin-2-yl)-nonanoic acid,
(4) 3(S)-(6-amino-pyridin-3-yl)-9-(5,6,7,8-tetrahydro-[1,8]-naphthyridin-2-yl)-nonanoic acid,
(5) 3(S)-(2-methyl-pyrimidin-5-yl)-9-(5,6,7,8-tetrahydro-[1,8]-naphthyridin-2-yl)-nonanoic acid,
(6) 3(S)-(quinolin-3-yl)-9-(5,6,7,8-tetrahydro-[1,8]-naphthyridin-2-yl)-nonanoic acid,
(7) 3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid,
(8) 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid,
(9) 3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid,
(10) 3(S)-(quinolin-3-yl)-3-{2-oxo-3-[3-(5,6,7,8-tetrahydro-[1,8]naphthyridin-2-yl)-propyl]-imidazolidin-1-yl}-propionic acid, and
(11) 3(S)-(4-ethoxy-3-fluorophenyl)-3-{2-oxo-3-[3-(5,6,7,8-tetrahydro-[1,8]-naphthyridin-2-yl)-propyl]-imidazolidin-1-yl}-propionic acid,
   or a pharmaceutically acceptable salt thereof, in combination with an antiinflammatory agent selected from the group consisting of:
(1) a salicylate, including acetylsalicylic acid,
(2) a non-steroidal antiinflammatory drug, including indomethacin, sulindac, mefenamic, meclofenamic, tolfenamic, tolmetin, ketorolac, dicofenac, ibuprofen, naproxen, fenoprofen, ketoprofen, flurbiprofin and oxaprozin,
(3) a corticosteroid, including dexamethasone and prednisolone
(4) a TNF inhibitor, including etanercept and infliximab,
(5) an IL-1 receptor antagonist,
(6) a cytotoxic or immunosuppressive drug, including methotrexate, leflunomide, azathioprine and cyclosporine,
(7) a gold compound,
(8) hydroxychloroquine or sulfasalazine,
(9) penicillamine,
(10) darbufelone, and
(11) a ρ38 kinase inhibitor,
   in an amount effective to treat or prevent the inflammatory disease or condition.

For purposes of this specification “halo” means F, Cl, Br, or I.

For purposes of this specification, “alkyl” means linear, branched and cyclic structures, and combinations thereof, containing the indicated number of carbon atoms. Examples of alkyl groups include methyl, ethyl, propyl, isopropyl, butyl, s- and t-butyl, pentyl, hexyl, heptyl, octyl, nonyl, undecyl, dodecyl, tridecyl, tetradecyl, pentadecyl, eicosyl, 3,7-diethyl-2,2-dimethyl-4-propylnonyl, cyclopropyl, cyclopentyl, cycloheptyl, adamantyl, cyclododecylmethyl, 2-ethyl-1-bicyclo[4.4.0]decyl and the like.

For purposes of this specification, “alkoxy” means alkoxy groups of the indicated number of carbon atoms of a straight, branched, or cyclic configuration. Examples of alkoxy groups include methoxy, ethoxy, propoxy, isopropoxy, cyclopropyloxy, cyclohexyloxy, and the like.

For purposes of this specification, the terms “inhibitor of cyclooxygenase-2,” “cyclooxygenase-2 specific inhibitor,” “cyclooxygenase-2 inhibitor” and “COX-2 inhibitor” as used herein embrace compounds which selectively inhibit cyclooxygenase-2 over cyclooxygenase-1. Employing the human whole blood COX-1 assay and the human whole blood COX-2 assay described in C. Brideau et al, Inflamm. Res. 45: 68-74 (1996), preferably, the compounds have a cyclooxygenase-2 IC₅₀ of less than about 2 uM in human whole blood COX-2 assay, yet have a cyclooxygenase-1 I₅₀ of greater than about 5 uM in the human whole blood COX-1 assay. Also preferably, the compounds have a selectivity ratio of cyclooxygenase-2 inhibition over cyclooxygenase-1 inhibition of at least 10, and preferably of at least 40. The resulting selectivity may indicate an ability to reduce the incidence of common NSAID-induced side effects.

Selective COX-2 inhibitors have been described in the scientific and patent literature, and reference is made to the following disclosures:
(1) J. Talley, Exp. Opin Ther. Patents, “Selective Inhibitors of cyclooxygenase-2,” Vol. 7, Issue 1, pp. 55-62 (1997).
(2) A. Kalgutkar, Exp. Opin Ther. Patents, “Selective Cyclooxygenase-2 inhibitors as non-ulcerogenic anti-inflammatory agents,” Vol. 9, Issue 7, pp. 831-849 (1999).
(3) P. Prasit and D. Riendeau, “Selective Cyclooxygenase-2 Inhibitors,” Annual Reports in Medicinal Chemistry, Volume 32, pp. 211-220 (1997).

Various structural classes of COX-2 inhibitors are disclosed in WO 99/10331, WO 98/21195, WO 98/05639, and U.S. Pat. No. 6,025,353 and U.S. Pat. No. 5,741,798.

Representative COX-2 inhibitors are also disclosed in the following patents. The compound 3-phenyl-4-(4-(methylsulfonyl)phenyl)-2-(5H)-furanone and similar COX-2 inhibitors are disclosed in U.S. Pat. No. 5,474,995, which is incorporated by reference in its entirety. Rofecoxib is the generic name for 3-phenyl-4-(4-(methylsulfonyl)phenyl)-2-(5H)-furanone.

The compound (5S)-ethyl-5-methyl-4-(4-(methanesulfonyl)phenyl)-3-(2-propoxy)-(5H)-furan-2-one and similar COX-2 inhibitors are disclosed in U.S. Pat. No. 6,020,343, which is incorporated by reference in its entirety.

The compound 5-chloro-3-(4-methylsulfonyl)phenyl-2-(2-methyl-5-pyridinyl)pyridine and similar COX-2 inhibitors are disclosed in U.S. Pat. No. 5,861,419, which is incorporated by reference in its entirety. Etoricoxib is the generic name for 5-chloro-3-(4-methylsulfonyl)phenyl-2-(2-methyl-5-pyridinyl)pyridine.

The compound 4-[5-(4-methylphenyl)-3-(trifluoromethyl)-1H-pyrazol-1-yl]benzenesulfonamide and similar COX-2 inhibitors are disclosed in U.S. Pat. No. 5,466,823, which is incorporated by reference in its entirety. Celecoxib is the generic name for 4-[5-(4-methylphenyl)-3-(trifluoromethyl)-1H-pyrazol-1-yl]benzenesulfonamide.

The compound 4-(5-methyl-3-phenyl-4isoxazolyl)-benzenesulfonamide and similar COX-2 inhibitors are disclosed in U.S. Pat. No. 5,633,272, which is incorporated by reference in its entirety. Valdecoxib is the generic name for 4-(5-Methyl-3-phenyl-4-isoxazolyl)-benzenesulfonamide.

The N-[[4-(5-methyl-3-phenyl-4-isoxazolyl)phenyl]sulfonyl]propanamide and similar COX-2 inhibitors are disclosed in U.S. Pat. No. 5,932,598, which is incorporated by reference herein in its entirety. Parecoxib is the generic name for N-[[4-(5-methyl-3-phenyl-4-isoxazolyl)phenyl]sulfonyl]propanamide.

For purposes of this specification, the term “integrin α_{V} antagonist” refers to the compounds that are antagonists of the integrin receptors α_{V}β₃, α_{V}β₅ and/or α_{V}β₆, such as those disclosed in U.S. Pat. Nos. 6,066,648, 6,048,861, 6,040,311 and 6,017,926, which are incorporated by reference in their entirety. Selective integrin α_{V} antagonists have been described, and reference is made to the following disclosure: G. Hartman and M. Duggan, “α_{V}β₃ Integrin Antagonists as Inhibitors of Bone Resorption,” Exp. Opin. Invest. Drugs Vol. 9, Issue 6, pp.1281-1291 (2000).

The representative integrin α_{V} antagonists are disclosed in the following patents. The integrin α_{V} antagonist 3(S)-(6-methoxypyridin-3-yl)-3-{2-oxo-3-[3-(5,6,7,8-tetrahydro-[1,8]-naphthyridin-2-yl)-propyl]imidazolidin-1-yl}-propionic acid and related integrin α_{V} antagonists are disclosed in U.S. Pat. No. 6,017,926. The integrin α_{V} antagonists 3(S)-(2-methyl-pyrimidin-5-yl)-9-(5,6,7,8-tetrahydro-[1,8]-naphthyridin-2-yl)-nonanoic acid; 3(S)-(pyrimidin-5-yl)-9-(5,6,7,8-tetrahydro-[1,8]-naphthyridin-2-yl)-nonanoic acid and related integrin α_{V} antagonists are dislosed in U.S. Pat. No. 6,048,861.

The instant combination of an integrin α_{V} antagonist and a cyclooxygenase-2 specific inhibitor is useful for the treatment of an inflammatory disease or condition. The combination of an integrin α_{V} antagonist and a cyclooxygenase-2specific inhibitor provides an unexpectedly superior effect in the treatment of an inflammatory disease or condition. When administered as part of a combination therapy, the cyclooxygenase-2 specific inhibitor together with the integrin α_{V} antagonist provides greater therapeutic efficacy with similar or fewer side effects as compared to the administration of either the cyclooxygenase-2 inhibitor or the integrin α_{V} antagonist alone.

In the combination of the present invention, the cyclooxygenase-2 specific inhibitor may be administered separately at different times during the course of therapy or in conjunction with the integrin α_{V} antagonist in divided or single combination forms. In addition, the administration of one element of the combination of the present invention may be prior to, concurrent to, or subsequent to the administration of the other element of the combination. The instant invention is therefore to be understood as embracing all such regimes of simultaneous or alternating treatment, and the term “administering” is to be interpreted accordingly. It will be understood that the scope of combinations of the compounds of this invention with other agents useful for treating integrin and cyclooxygenase-mediated conditions includes in principle any combination with any pharmaceutical composition useful for treating or preventing an inflammatory disease or condition.

The term integrin α_{V} antagonist is intended to include all pharmaceutically acceptable salt forms of compounds that have α_{V}β₃, α_{V}β₅ and/or α_{V}β₆ integrin receptor antagonist activity, and therefore the use of such salts is included within the scope of this invention. For use in medicine, the salts of the compounds of this invention refer to non-toxic “pharmaceutically acceptable salts.” Other salts may, however, be useful in the preparation of the compounds according to the invention or of their pharmaceutically acceptable salts. Salts encompassed within the term “pharmaceutically acceptable salts” refer to non-toxic salts of the compounds of this invention which are generally prepared by reacting the free base with a suitable organic or inorganic acid. Representative salts include the following:

Acetate, Benzenesulfonate, Benzoate, Bicarbonate, Bisulfate, Bitartrate, Borate, Bromide, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Sulfate, Subacetate, Succinate, Tannate, Tartrate, Teoclate, Tosylate, Triethiodide and Valerate.

Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds of the present invention may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers. Also included within the scope of the invention are polymorphs and hydrates of the compounds of the instant invention.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of an inflammatory disease or condition with the compound specifically disclosed as an element of the combination or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985.

The term “amount effective to treat or prevent” shall mean that amount of a drug or pharmaceutical agent that will elicit the biological or medical response of a tissue, system, animal or human that is being sought by a researcher or clinician.

The elements of the combination of the present invention may be administered by oral, parenteral (e.g., intramuscular, intraperitoneal, intravenous or subcutaneous injection, or implant), buccal, nasal, vaginal, rectal, sublingual, or topical (e.g., ocular eyedrop) routes of administration and may be formulated, alone or together, in suitable dosage unit formulations containing conventional non-toxic pharmaceutically acceptable carriers, adjuvants and vehicles appropriate for each route of administration.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in the combination in an amount sufficient to produce the desired pharmacologic effect upon the process or condition of inflammation.

The pharmaceutical compositions containing the active ingredient suitable for oral administration may be in the form of discrete units such as hard or soft capsules, tablets, troches or lozenges, each containing a predetermined amount of the active ingredient; in the form of a dispersible powder or granules; in the form of a solution or a suspension in an aqueous liquid or non-aqueous liquid; in the form of syrups or elixirs; or in the form of an oil-in-water emulsion or a water-in-oil emulsion. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide a pharmaceutically elegant and palatable preparation.

Solid dosage forms for oral administration include capsules, tablets, pills, powders and granules. In such solid dosage forms, the active compounds are admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. In the case of capsules, tablets and pills, the dosage forms may also comprise buffering agents.

Tablets containing the active ingredient in admixture with non-toxic pharmaceutically acceptable excipients may also be manufactured by known methods. The excipients used may be for example, (1) inert diluents such as calcium carbonate, lactose, calcium phosphate or sodium phosphate; (2) granulating and disintegrating agents, such as corn starch or alginic acid; (3) binding agents such as starch, gelatin or acacia; and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,160,452; and 4,265,874 to form osmotic therapeutic tablets for controlled release.

In some cases, formulations for oral use may be in the form of hard gelatin capsules wherein the active ingredient is mixed with an inert solid diluent, for example calcium carbonate, calcium phosphate or kaolin. They may also be in the form of soft gelatin capsules wherein the active ingredient is mixed with water or an oil medium, for example peanut oil, liquid paraffin, or olive oil.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants, such as wetting agents, emulsifying and suspending agents, and sweetening, flavoring, and perfuming agents.

Aqueous suspensions normally contain the active materials in admixture with excipients suitable for the manufacture of aqueous suspensions. Such excipients may be
1) suspending agents such as sodium carboxymethyl-cellulose, methylcellulose, hydroxypropylmethyl-cellulose, sodium alginate, polyvinyl-pyrrolidone, gum tragacanth and gum acacia;
(2) dispersing or wetting agents which may be
   (a) a naturally-occurring phosphatide such as lecithin,
   (b) a condensation product of an alkylene oxide with a fatty acid, for example, polyoxyethylene stearate,
   (c) a condensation product of ethylene oxide with a long chain aliphatic alcohol, for example, heptadecaethyleneoxycetanol,
   (d) a condensation product of ethylene oxide with a partial ester derived from a fatty acid and a hexitol such as polyoxyethylene sorbitol monooleate, or
   (e) a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions of the invention may also be in the form of oil-in-water emulsions. The oily phase may be a vegetable oil such as olive oil or arachis oils, or a mineral oil such as liquid paraffin or a mixture thereof. Suitable emulsifying agents may be (1) naturally-occurring gums such as gum acacia and gum tragacanth, (2) naturally-occurring phosphatides such as soybean and lecithin, (3) esters or partial esters derived from fatty acids and hexitol anhydrides, for example, sorbitan monooleate, (4) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension or solution. The suspension may be formulated according to known methods using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane-diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Preparations according to this invention for parenteral administration include sterile aqueous or non-aqueous solutions, suspension, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use. The combination of this invention may also be administered in the form of suppositories for rectal administration. This composition can be prepared by mixing the drug with a suitable non-irritating excipient which is solid at ordinary temperatures but liquid at the rectal temperature and will therefore melt in the rectum to release the drug. Such materials are cocoa butter and polyethylene glycols. Compositions for buccal, nasal or sublingual administration are also prepared with standard excipients well known in the art.

For topical administration the combination of this invention may be formulated in liquid or semi-liquid preparations such as liniments, lotions, applications; oil-in-water or water-in-oil emulsions such as creams, ointments, jellies or pastes, including tooth-pastes; or solutions or suspensions such as drops, mouthwashes, and the like.

The dosage of the active ingredients in the compositions of this invention may be varied. However, it is necessary that the amount of the active ingredient be such that a suitable dosage form is obtained. The selected dosage depends upon the desired therapeutic effect, on the route of administration and on the duration of the treatment. Generally, dosage levels of the cyclooxygenase-2 specific inhibitor are between about 0.001 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, 250 and 500 milligrams of each of the active ingredients for the symptomatic adjustment of the dosage to the patient to be treated. A medicament typically contains from about 0.01 mg to about 500 mg of each of the active ingredients, preferably, from about 1 mg to about 100 mg of each of the active ingredients. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Dosage levels of the integrin α_{V} antagonist of between about 0.001 to 50 mg/kg of body weight daily, preferably about 0.005 to about 25 mg/kg per day, and more preferably about 0.01 to about 10 mg/kg per day are administered to a patient to obtain effective treatment of an inflammatory disease or condition.

An especially preferred combination is that wherein the cycloxygenase-2 specific inhibitor is administered at a dosage rate of about 0.01 to about 10 mg/kg/day, especially about 0.05 to about 5.0 mg/kg/day, and more particularly about 0.1 to about 5 mg/kg/day, and the integrin α_{V} antagonist is administered at a dosage level of about 0.001 to about 20 mg/kg/day, especially about 0.005 to about 10 mg/kg/day, and more particularly about 0.01 to about 5 mg/kg/day.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Combinations of the present invention are useful for the treatment or prevention of an inflammatory disease or condition. For example, combinations of the present invention would be useful to treat arthritis, including but not limited to rheumatoid arthritis, osteoarthritis, ankylosing spondylitis, gout and juvenile arthritis.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the patient being treated for an inflammatory disease or condition. Likewise, the specific pharmacological responses observed may vary according to and depending upon the particular active compound or combination selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method for treating or preventing an inflammatory disease or condition in a mammalian patient in need of such treatment comprising administering to said patient an integrin α_{V} antagonist in combination with a cyclooxygenase-2 specific inhibitor in an amount that is effective to treat or prevent the inflammatory disease or condition.

2. The method according to claim 1 wherein the cyclooxygenase-2 specific inhibitor is selected from the group consisting of:
(1) 3-phenyl-4-(4-(methylsulfonyl)phenyl)-2-(5H)-furanone;
(2) 3-(3-fluorophenyl)-4-(4-methylsulfonyl)phenyl)-2-(5H)-furanone;
(3) 3-(3,4-difluorophenyl)-4-(4-(methylsulfonyl)phenyl)-2-(5H)-furanone;
(4) 3-(3,4-trichlorophenyl)-4-(4-(methylsulfonyl)phenyl)-2-(5H)-furanone;
(5) 3-(3,4-dichlorophenyl)-4-(4-(aminosulfonyl)phenyl)-2-(5H)-furanone;
(6) 3-(3-chloro-4-methoxyphenyl)-4-(4-aminosulfonyl)phenyl)-2-(5H)-furanone;
(7) 4-[5-(4-methylphenyl)-3-(trifluoromethyl)-1H-pyrazol-1-yl]benzenesulfonamide;
(8) (5S)-ethyl-5-methyl-4-(4-(methanesulfonyl)phenyl)-3-(2-propoxy)-(5H)-furan-2-one;
(9) 5,5-dimethyl-4-(4-(methylsulfonyl)phenyl)-3-(2-propoxy)-5H-furan-2-one;
(10) 5,5-dimethyl-4-(4-(methylsulfonyl)phenyl)-3-(5-bromopyridin-2-yloxy)-5H-furan-2-one;
(11) 5-methyl-4-(4-methylsulfonyl)phenyl)-3-(2-(propoxy)-5-(2-trifluoroethyl)-5H-furan-2-one;
(12) 3-(3-trifluoromethyl)phenoxy-4-(4-methylsulfonyl)phenyl)-5,5-dimethyl-5H-furan-2-one;
(13) (5R)-3-(3-chloro-4-methoxyphenoxy)-5-ethyl-5-methyl-4-(4-methylsulfonyl)phenyl-5H-furan-2-one;
(14) 5-chloro-3-(4-methylsulfonyl)phenyl-2-(2-methyl-5-pyridinyl)pyridine;
(15) 5-chloro-3-(4-methylsulfonyl)phenyl-2-(2-ethyl-5-pyridinyl)pyridine;
(16) 5-chloro-3-(4-(methylsulfonyl)phenyl)-2-(3-pyridinyl)pyridine;
(17) 4-(5-methyl-3-phenyl-4-isoxazolyl)-benzenesulfonamide; and
(18) N-[[4-(5-methyl-3-phenyl-4-isoxazolyl)phenyl]sulfonyl]propanamide;
or a pharmaceutically acceptable salt thereof.

3. The method according to claim 2 wherein the cyclooxygenase-2 specific inhibitor is selected from the group consisting of:
(1) 3-phenyl-4-(4-(methylsulfonyl)phenyl)-2-(5H)-furanone;
(2) 4-[5-(4-methylphenyl)-3-(trifluoromethyl)-1H-pyrazol-1-yl]benzenesulfonamide;
(3) 4-(5-methyl-3-phenyl-4-isoxazolyl)-benzenesulfonamide;
(4) N-[[4-(5-methyl-3-phenyl-4-isoxazolyl)phenyl]sulfonyl]propanamide;
(5) 5-chloro-3-(4-methylsulfonyl)phenyl-2-(2-methyl-5-pyridinyl)pyridine; and
(6) (5S)-ethyl-5-methyl-4-(4-(methanesulfonyl)phenyl)-3-(2-propoxy)-(5H)-furan-2-one;
or a pharmaceutically acceptable salt thereof.

4. The method according to claim 1 wherein the integrin α_{V} antagonist is a compound of formula I 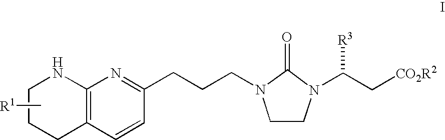
or a pharmaceutically acceptable salt thereof, wherein:
R¹ is hydrogen or C₁₋₄ alkyl;
R² is hydrogen or C₁₋₄ alkyl; and
R³ is selected from the group consisting of: dihydrobenzofuranyl, phenyl, quinolinyl and pyridinyl, each optionally substituted with 1-2 substituents independently selected from the group consisting of: halo, hydroxy, cyano, C₁₋₆alkyl, C₁₋₃alkoxy, amino, C₁₋₃ alkylamino and di(C₁₋₃) alkylamino, said C₁₋₆alkyl and C₁₋₃alkoxy each optionally substituted with 1-3 halo groups.

5. The method according to claim 4 wherein the integrin α_{V} antagonist is selected from the group consisting of:
(1) 3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;
(2) 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid;
(3) 3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid;
(4) 3(S)-(quinolin-3-yl)-3-{2-oxo-3-[3-(5,6,7,8-tetrahydro-[1,8]naphthyridin-2-yl)-propyl]-imidazolidin-1-yl}-propionic acid; and
(5) 3(S)-(4-ethoxy-3-fluorophenyl)-3-{2-oxo-3-[3-(5,6,7,8-tetrahydro-[1,8]-naphthyridin-2-yl)-propyl]-imidazolidin-1-yl}-propionic acid;
or a pharmaceutically acceptable salt thereof.

6. The method according to claim 5 wherein the cyclooxygenase-2 specific inhibitor is selected from the group consisting of:
(1) 3-phenyl-4-(4-(methylsulfonyl)phenyl)-2-(5H)-furanone;
(2) 4-[5-(4-methylphenyl)-3-(trifluoromethyl)-1H-pyrazol-1-yl]benzenesulfonamide;
(3) 4-(5-methyl-3-phenyl-4-isoxazolyl)-benzenesulfonamide;
(4) N-[[4-(5-methyl-3-phenyl-4-isoxazolyl)phenyl]sulfonyl]propanamide;
(5) 5-chloro-3-(4-methylsulfonyl)phenyl-2-(2-methyl-5-pyridinyl)pyridine; and
(6) (5S)-ethyl-5-methyl-4-(4-(methanesulfonyl)phenyl)-3-(2-propoxy)-(5H)-furan-2-one;
or a pharmaceutically acceptable salt thereof.

7. The method according to claim 5 wherein the integrin α_{V} antagonist is 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid or a pharmaceutically acceptable salt thereof.

8. The method according to claim 7 wherein the cyclooxygenase-2 specific inhibitor is selected from the group consisting of: 3-phenyl4-(4-(methylsulfonyl)phenyl)-2-(5H)-furanone, 5-chloro-3-(4-methylsulfonyl)phenyl-2-(2-methyl-5-pyridinyl)pyridine and (5S)-ethyl-5-methyl-4-(4-(methanesulfonyl)phenyl)-3-(2-propoxy)-(5H)-furan-2-one, or a pharmaceutically acceptable salt thereof.

9. The method according to claim 1 wherein the integrin α_{V} antagonist is a compound of formula II 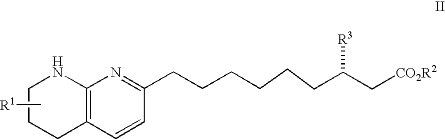
or a pharmaceutically acceptable salt thereof, wherein:
R¹ is hydrogen or C₁₋₄ alkyl;
R² is hydrogen or C₁₋₄ alkyl; and
R³ is selected from the group consisting of: quinolinyl, pyridinyl and pyrimidinyl, each optionally substituted with 1-2 substituents independently selected from the group consisting of: halo, hydroxy, cyano, C₁₋₆alkyl, C₁₋₃alkoxy, amino, C₁₋₃ alkylamino and di(C₁₋₃)alkylamino, said C₁₋₆alkyl and C₁₋₃alkoxy each optionally substituted with 1-3 halo groups.

10. The method according to claim 9 wherein the integrin α_{V} antagonist is selected from the group consisting of:
(1) 3-(pyridin-3-yl)-9-(5,6,7,8-tetrahydro-[1,8]-naphthyridin-2-yl)-nonanoic acid;
(2) 3(S)-(6-methoxy-pyridin-3-yl)-9-(5,6,7,8-tetrahydro-[1,8]-naphthyridin-2-yl)-nonanoic acid;
(3) 3(S)-(pyrimidin-5-yl)-9-(5,6,7,8-tetrahydro-[1,8]-naphthyridin-2-yl)-nonanoic acid;
(4) 3(S)-(6-amino-pyridin-3-yl)-9-(5,6,7,8-tetrahydro-[1,8]-naphthyridin-2-yl)-nonanoic acid;
(5) 3(S)-(2-methyl-pyrimidin-5-yl)-9-(5,6,7,8-tetrahydro-[1,8)-naphthyridin-2-yl)-nonanoic acid; and
(6) 3(S)-(quinolin-3-yl)-9-(5,6,7,8-tetrahydro-[1,8]-naphthyridin-2-yl)-nonanoic acid;
or a pharmaceutically acceptable salt thereof.

11. The method according to claim 10 wherein the cyclooxygenase-2 specific inhibitor is selected from the group consisting of:
(1) 3-phenyl-4-(4-(methylsulfonyl)phenyl)-2-(5H)-furanone;
(2) 4-[5-(4-methylphenyl)-3-(trifluoromethyl)-1H-pyrazol-1-yl]benzenesulfonamide;
(3) 4-(5-methyl-3-phenyl-4-isoxazolyl)-benzenesulfonamide;
(4) N-[[4-(5-methyl-3-phenyl-4-isoxazolyl)phenyl]sulfonyl]propanamide;
(5) 5-chloro-3-(4-methylsulfonyl)phenyl-2-(2-methyl-5-pyridinyl)pyridine; and
(6) (5S)-ethyl-5-methyl-4-(4-(methanesulfonyl)phenyl)-3-(2-propoxy)-(5H)-furan-2-one;
or a pharmaceutically acceptable salt thereof.

12. The method according to claim 10 wherein the integrin α_{V} antagonist is selected from the group consisting of: 3(S)-(pyrimidin-5-yl)-9-(5,6,7,8-tetrahydro-[1,8]-naphthyridin-2-yl)-nonanoic acid and 3(S)-(2-methyl-pyrimidin-5-yl)-9-(5,6,7,8-tetrahydro-[1,8]-naphthyridin-2-yl)-nonanoic acid, or a pharmaceutically acceptable salt thereof.

13. The method according to claim 12 wherein the cyclooxygenase-2 specific inhibitor is selected from the group consisting of: 3-phenyl-4-(4-(methylsulfonyl)phenyl)-2-(5H)-furanone, 5-chloro-3-(4-methylsulfonyl)phenyl-2-(2-methyl-5-pyridinyl)pyridine and (5S)-ethyl-5-methyl4-(4-(methanesulfonyl)phenyl)-3-(2-propoxy)-(5H)-furan-2-one, or a pharmaceutically acceptable salt thereof.

14. The method according to claim 1 wherein the integrin α_{V} antagonist is an α_{V}β₃ integrin receptor antagonist.

15. The method according to claim 1 wherein the integrin α_{V} antagonist is an α_{V}β₅ integrin receptor antagonist.

16. The method according to claim 1 wherein the integrin α_{V} antagonist is an α_{V}β₆ integrin receptor antagonist.

17. The method according to claim 1 wherein the integrin α_{V} antagonist is a dual α_{V}β₃/α_{V}β₅ integrin receptor antagonist.

18. The method according to claim 1 wherein the integrin α_{V} antagonist is a mixed α_{V}β₃, α_{V}β₅ and α_{V}β₆ integrin receptor antagonist.

19. The use of an integrin α_{V} antagonist in combination with a cyclooxygenase-2 specific inhibitor for the preparation of a medicament useful for the treatment or prevention of an inflammatory disease or condition.

20. The method according to claim 1 wherein the inflammatory disease or condition is rheumatoid arthritis.

21. The method according to claim 20 wherein the cyclooxygenase-2 specific inhibitor is selected from the group consisting of:
(1) 3-phenyl-4-(4-(methylsulfonyl)phenyl)-2-(5H)-furanone;
(2) 4-[5-(4-methylphenyl)-3-(trifluoromethyl)-1H-pyrazol-1-yl]benzenesulfonamide;
(3) 4-(5-methyl-3-phenyl-4-isoxazolyl)-benzenesulfonamide;
(4) N-[[4-(5-methyl-3-phenyl-4-isoxazolyl)phenyl]sulfonyl]propanamide;
(5) 5-chloro-3-(4-methylsulfonyl)phenyl-2-(2-methyl-5-pyridinyl)pyridine; and
(6) (5S)-ethyl-5-methyl-4-(4-(4-(methanesulfonyl)phenyl)-3-(2-propoxy)-(5H)-furan-2-one;
or a pharmaceutically acceptable salt thereof.

22. The method according to claim 20 wherein the integrin α_{V} antagonist is selected from the group consisting of:
(1) 3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;
(2) 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid;
(3) 3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid;
(4) 3(S)-(quinolin-3-yl)-3-{2-oxo-3-[3-(5,6,7,8-tetrahydro-[1,8]naphthyridin-2-yl)-propyl]-imidazolidin-1-yl}-propionic acid;
(5) 3(S)-(4-ethoxy-3-fluorophenyl)-3-{2-oxo-3-[3-(5,6,7,8-tetrahydro-[1,8]-naphthyridin-2-yl)-propyl]-imidazolidin-1-yl}-propionic acid;
(6) 3-(pyridin-3-yl)-9-(5,6,7,8-tetrahydro-[1,8]-naphthyridin-2-yl)-nonanoic acid;
(7) 3(S)-(6-methoxy-pyridin-3-yl)-9-(5,6,7,8-tetrahydro-[1,8]-naphthyridin-2-yl)-nonanoic acid;
(8) 3(S)-(pyrimidin-5-yl)-9-(5,6,7,8-tetrahydro-[1,8]-naphthyridin-2-yl)-nonanoic acid;
(9) 3(S)-(6-amino-pyridin-3-yl)-9-(5,6,7,8-tetrahydro-[1,8]-naphthyridin-2-yl)-nonanoic acid;
(10) 3(S)-(2-methyl-pyrimidin-5-yl)-9-(5,6,7,8-tetrahydro-[1,8]-naphthyridin-2-yl)-nonanoic acid; and
(11) 3(S)-(quinolin-3-yl)-9-(5,6,7,8-tetrahydro-[1,8]-naphthyridin-2-yl)-nonanoic acid;
or a pharmaceutically acceptable salt thereof.

23. The method according to claim 1 wherein the inflammatory disease or condition is osteoarthritis.

24. The method according to claim 23 wherein the cyclooxygenase-2 specific inhibitor is selected from the group consisting of:
(1) 3-phenyl-4-(4-(methylsulfonyl)phenyl)-2-(5H)-furanone;
(2) 4-[5-(4-methylphenyl)-3-(trifluoromethyl)-1H-pyrazol-1-yl]benzenesulfonamide;
(3) 4-(5-methyl-3-phenyl4-isoxazolyl)-benzenesulfonamide;
(4) N-[[4-(5-methyl-3-phenyl-4-isoxazolyl)phenyl]sulfonyl]propanamide;
(5) 5-chloro-3-(4-methylsulfonyl)phenyl-2-(2-methyl-5-pyridinyl)pyridine; and
(6) (5S)-ethyl-5-methyl-4-(4-(methanesulfonyl)phenyl)-3-(2-propoxy)-(5H)-furan-2-one;
or a pharmaceutically acceptable salt thereof.

25. The method according to claim 23 wherein the integrin α_{V} antagonist is selected from the group consisting of:
(1) 3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;
(2) 3(S)-(6-methoxypyridin-3-yl)-3-{2-oxo-3-[3-(5,6,7,8-tetrahydro-[1,8)-naphthyridin-2-yl)-propyl]-imidazolidin-1-yl}-propionic acid;
(3) 3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid;
(4) 3(S)-(quinolin-3-yl)-3-{2-oxo-3-[3-(5,6,7,8-tetrahydro-[1,8]naphthyridin-2-yl)-propyl]-imidazolidin-1-yl}-propionic acid;
(5) 3(S)-(4ethoxy-3-fluorophenyl)-3-{2-oxo-3-[3-(5,6,7,8-tetrahydro-[1,8]-naphthyridin-2-yl)-propyl]-imidazolidin-1-yl}-propionic acid;
(6) 3-(pyridin-3-yl)-9-(5,6,7,8-tetrahydro-[1,8]-naphthyridin-2-yl)-nonanoic acid;
(7) 3(S)-(6-methoxy-pyridin-3-yl)-9-(5,6,7,8-tetrahydro-[1,8]-naphthyridin-2-yl)-nonanoic acid;
(8) 3(S)-(pyrimidin-5-yl)-9-(5,6,7,8-tetrahydro-[1,8]-naphthyridin-2-yl)-nonanoic acid;
(9) 3(S)-(6-amino-pyridin-3-yl)-9-(5,6,7,8-tetrahydro-[1,8]-naphthyridin-2-yl)-nonanoic acid;
(10) 3(S)-(2-methyl-pyrimidin-5-yl)-9-(5,6,7,8-tetrahydro-[1,8]-naphthyridin-2-yl)-nonanoic acid; and
(11) 3(S)-(quinolin-3-yl)-9-(5,6,7,8-tetrahydro-[1,8]-naphthyridin-2-yl)-nonanoic acid;
or a pharmaceutically acceptable salt thereof.

26. A pharmaceutical composition comprising a pharmaceutically acceptable carrier, a cyclooxygenase-2 specific inhibitor and an integrin α_{V} antagonist of formula I 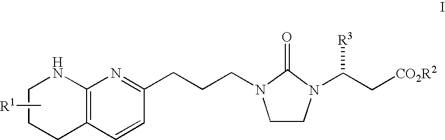
or a pharmaceutically acceptable salt thereof, wherein:
R¹ is hydrogen or C₁₋₄ alkyl;
R² is hydrogen or C₁₋₄ alkyl; and
R³ is selected from the group consisting of: dihydrobenzofuranyl, phenyl, quinolinyl and pyridinyl, each optionally substituted with 1-2 substituents independently selected from the group consisting of: halo, hydroxy, cyano, C₁₋₆alkyl, C₁₋₃alkoxy, amino, C₁₋₃ alkylamino and di(C₁₋₃)alkylamino, said C₁₋₆alkyl and C₁₋₃alkoxy each optionally substituted with 1-3 halo groups.

27. The pharmaceutical composition according to claim 26 wherein the cyclooxygenase-2 specific inhibitor is selected from the group consisting of:
(1) 3-phenyl-4-(4-(methylsulfonyl)phenyl)-2-(5H)-furanone;
(2) 3-(3-fluorophenyl)-4-(4-methylsulfonyl)phenyl)-2-(5H)-furanone;
(3) 3-(3,4-difluorophenyl)-4-(4-(methylsulfonyl)phenyl)-2-(5H)-furanone;
(4) 3-(3,4-trichlorophenyl)-4-(4-(methylsulfonyl)phenyl)-2-(5H)-furanone;
(5) 3-(3,4-dichlorophenyl)-4-(4-(aminosulfonyl)phenyl)-2-(5H)-furanone;
(6) 3-(3-chloro-4-methoxyphenyl)-4-(4-aminosulfonyl)phenyl)-2-(5H)-furanone;
(7) 4-[5-(4-methylphenyl)-3-(trifluoromethyl)-1H-pyrazol-1-yl]benzenesulfonamide;
(8) (5S)-ethyl-5-methyl-4-(4-(methanesulfonyl)phenyl)-3-(2-propoxy)-(5H)-furan-2-one;
(9) 5,5-dimethyl-4-(4-(methylsulfonyl)phenyl)-3-(2-propoxy)-5H-furan-2-one;
(10) 5,5-dimethyl-4-(4-(methylsulfonyl)phenyl)-3-(5-bromopyridin-2-yloxy)-5H-furan-2-one;
(11) 5-methyl-4-(4-methylsulfonyl)phenyl)-3-(2-(propoxy)-5-(2-trifluoroethyl)-5H-furan-2-one;
(12) 3-(3-trifluoromethyl)phenoxy-4-(4-methylsulfonyl)phenyl)-5,5-dimethyl-5H-furan-2-one;
(13) (5R)-3-(3-chloro-4-methoxyphenoxy)-5-ethyl-5-methyl-4-(4-methylsulfonyl)phenyl-5H-furan-2-one;
(14) 5-chloro-3-(4-methylsulfonyl)phenyl-2-(2-methyl-5-pyridinyl)pyridine;
(15) 5-chloro-3-(4-methylsulfonyl)phenyl-2-(2-ethyl-5-pyridinyl)pyridine;
(16) 5-chloro-3-(4-(methylsulfonyl)phenyl)-2-(3-pyridinyl)pyridine;
(17) 4-(5-methyl-3-phenyl-4-isoxazolyl)-benzenesulfonamide; and
(18) N-[[4-(5-methyl-3-phenyl-4-isoxazolyl)phenyl]sulfonyl]propanamide;
or a pharmaceutically acceptable salt thereof.

28. The pharmaceutical composition according to claim 26 wherein the integrin α_{V} antagonist is selected from the group consisting of:
(1) 3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;
(2) 3(S)-(6-methoxypyridin-3-yl)-3-{2-oxo-3-[3-(5,6,7,8-tetrahydro-[1,8)-naphthyridin-2-yl)-propyl]-imidazolidin-1-yl}-propionic acid;
(3) 3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid;
(4) 3(S)-(quinolin-3-yl)-3-{2-oxo-3-[3-(5,6,7,8-tetrahydro-[1,8]naphthyridin-2-yl)-propyl]-imidazolidin-1-yl}-propionic acid; and
(5) 3(S)-(4-ethoxy-3-fluorophenyl)-3-{2-oxo-3-[3-(5,6,7,8-tetrahydro-[1,8]-naphthyridin-2-yl)-propyl]-imidazolidin-1-yl}-propionic acid;
or a pharmaceutically acceptable salt thereof.

29. The pharmaceutical composition according to claim 28 wherein the integrin α_{V} antagonist is 3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid or a pharmaceutically acceptable salt thereof.

30. The pharmaceutical composition according to claim 29 wherein the cyclooxygenase-2 specific inhibitor is selected from the group consisting of: 3-phenyl-4-(4-(methylsulfonyl)phenyl)-2-(5H)-furanone, 5-chloro-3-(4-methylsulfonyl)phenyl-2-(2-methyl-5-pyridinyl)pyridine, and (5S)-ethyl-5-methyl-4-(4-(methanesulfonyl)phenyl)-3-(2-propoxy)-(5H)-furan-2-one, or a pharmaceutically acceptable salt thereof.

31. A pharmaceutical composition comprising a pharmaceutically acceptable carrier, a cyclooxygenase-2 specific inhibitor and an integrin α_{V} antagonist of formula II 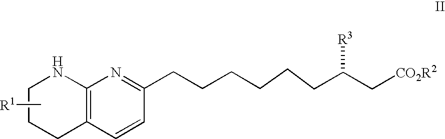
or a pharmaceutically acceptable salt thereof, wherein:
R¹ is hydrogen or C₁₋₄ alkyl;
R² is hydrogen or C₁₋₄ alkyl; and
R³ is selected from the group consisting of: quinolinyl, pyridinyl and pyrimidinyl, each optionally substituted with 1-2 substituents independently selected from the group consisting of: halo, hydroxy, cyano, C₁₋₆alkyl, C₁₋₃alkoxy, amino, C₁₋₃ alkylamino and di(C₁₋₃)alkylamino, said C₁₋₆alkyl and C₁₋₃alkoxy each optionally substituted with 1-3 halo groups.

32. The pharmaceutical composition according to claim 31 wherein the cyclooxygenase-2 specific inhibitor is selected from the group consisting of:
(1) 3-phenyl-4-(4-(methylsulfonyl)phenyl)-2-(5H)-furanone;
(2) 3-(3-fluorophenyl)-4-(4-methylsulfonyl)phenyl)-2-(5H)-furanone;
(3) 3-(3,4-difluorophenyl)-4-(4-(methylsulfonyl)phenyl)-2-(5H)-furanone;
(4) 3-(3,4-trichlorophenyl)-4-(4-(methylsulfonyl)phenyl)-2-(5H)-furanone;
(5) 3-(3,4-dichlorophenyl)-4-(4-(aminosulfonyl)phenyl)-2-(5H)-furanone;
(6) 3-(3-chloro-4-methoxyphenyl)-4-(4-aminosulfonyl)phenyl)-2-(5H)-furanone;
(7) 4-[5-(4-methylphenyl)-3-(trifluoromethyl)-1H-pyrazol-1-yl]benzenesulfonamide;
(8) (5S)-ethyl-5-methyl-4-(4-(methanesulfonyl)phenyl)-3-(2-propoxy)-(5H)-furan-2-one;
(9) 5,5-dimethyl4-(4-(methylsulfonyl)phenyl)-3-(2-propoxy)-5H-furan-2-one;
(10) 5,5-dimethyl-4-(4-(methylsulfonyl)phenyl)-3-(5-bromopyridin-2-yloxy)-5H-furan-2-one;
(11) 5-methyl-4-(4-methylsulfonyl)phenyl)-3-(2-(propoxy)-5-(2-trifluoroethyl)-5H-furan-2-one;
(12) 3-(3-trifluoromethyl)phenoxy-4-(4-methylsulfonyl)phenyl)-5,5-dimethyl-5H-furan-2-one;
(13) (5R)-3-(3-chloro-4-methoxyphenoxy)-5-ethyl-5-methyl-4-(4-methylsulfonyl)phenyl-5H-furan-2-one;
(14) 5-chloro-3-(4-methylsulfonyl)phenyl-2-(2-methyl-5-pyridinyl)pyridine;
(15) 5-chloro-3-(4-methylsulfonyl)phenyl-2-(2-ethyl-5-pyridinyl)pyridine;
(16) 5-chloro-3-(4-(methylsulfonyl)phenyl)-2-(3-pyridinyl)pyridine;
(17) 4-(5-methyl-3-phenyl-4-isoxazolyl)-benzenesulfonamide; and
(18) N-[[4-(5-methyl-3-phenyl-4-isoxazolyl)phenyl]sulfonyl]propanamide;
or a pharmaceutically acceptable salt thereof.

33. The pharmaceutical composition according to claim 31 wherein the integrin α_{V} antagonist is selected from the group consisting of:
(1) 3-(pyridin-3-yl)-9-(5,6,7,8-tetrahydro-[1,8]-naphthyridin-2-yl)-nonanoic acid;
(2) 3(S)-(6-methoxy-pyridin-3-yl)-9-(5,6,7,8-tetrahydro-[1,8]-naphthyridin-2-yl)-nonanoic acid;
(3) 3(S)-(pyrimidin-5-yl)-9-(5,6,7,8-tetrahydro-[1,8]-naphthyridin-2-yl)-nonanoic acid;
(4) 3(S)-(6-amino-pyridin-3-yl)-9-(5,6,7,8-tetrahydro-[1,8]-naphthyridin-2-yl)-nonanoic acid;
(5) 3(S)-(2-methyl-pyrimidin-5-yl)-9-(5,6,7,8-tetrahydro-[1,8]-naphthyridin-2-yl)-nonanoic acid; and
(6) 3(S)-(quinolin-3-yl)-9-(5,6,7,8-tetrahydro-[1,8]-naphthyridin-2-yl)-nonanoic acid;
or a pharmaceutically acceptable salt thereof.

34. The pharmaceutical composition according to claim 33 wherein the integrin α_{V} antagonist is selected from the group consisting of: 3(S)-(pyrimidin-5-yl)-9-(5,6,7,8-tetrahydro-[1,8]-naphthyridin-2-yl)-nonanoic acid and 3(S)-(2-methyl-pyrimidin-5-yl)-9-(5,6,7,8-tetrahydro-[1,8]-naphthyridin-2-yl)-nonanoic acid, or a pharmaceutically acceptable salt thereof.

35. The pharmaceutical composition according to claim 34 wherein the cyclooxygenase-2 specific inhibitor is selected from the group consisting of: 3-phenyl-4-(4-(methylsulfonyl)phenyl)-2-(5H)-furanone, 5-chloro-3-(4-methylsulfonyl)phenyl-2-(2-methyl-5-pyridinyl)pyridine, and (5S)-ethyl-5-methyl-4-(4-(methanesulfonyl)phenyl)-3-(2-propoxy)-(5H)-furan-2-one, or a pharmaceutically acceptable salt thereof.
